(19) United States
(12) Patent Application Publication
(10) Pub. No.: US 2015/0190555 A1
(43) Pub. Date: Jul. 9, 2015

(54) Methods, Systems, and Devices Relating to Directional Eluting Implantable Medical Devices

(71) Applicant: South Dakota Board of Regents, Pierre, SD (US)

(72) Inventor: Gopinath Mani, Sioux Falls, SD (US)

(21) Appl. No.: 14/418,635

(22) PCT Filed: Jul. 17, 2013
(86) PCT No.: PCT/US13/50904

§ 371 Date: Jan. 30, 2015

Related U.S. Application Data
(60) Provisional application No. 61/679,955, filed on Aug. 6, 2012.

(51) Int. Cl. A61L 31/16; A61F 2/82; A61L 31/12; A61F 2/07

(52) U.S. Cl. CPC A61L 31/16; A61F 2/07; A61F 2/82; A61L 31/12; A61F 2240/001; A61F 2250/0067; A61L 2300/216; A61L 2300/114; A61L 2420/00; A61L 2400/18

(57) ABSTRACT

Implantable medical devices may directionally elute a first therapeutic agent that promotes the growth of endothelial cells and a second therapeutic agent that inhibits the growth of smooth muscle cells. In some embodiments, implantable medical devices may elute a first therapeutic agent such as an anti-proliferative drug from an abluminal side of the implantable medical device and a second therapeutic agent such as an endothelialization agent from a luminal side of the implantable medical device.

### CROSS-REFERENCE TO RELATED APPLICATIONS

This application claims priority from U.S. Provisional Application 61/679,955, filed Aug. 6, 2012, and entitled “Directional Eluting Implantable Medical Devices,” which is hereby incorporated herein by reference in its entirety.

### FIELD OF THE INVENTION

The application pertains generally to implantable medical devices and more particularly to implantable medical devices that provide directional elution of one or more therapeutic agents.

### BACKGROUND OF THE INVENTION

Coronary artery disease (CAD) is the leading cause of death in the United States for both men and women. This disease is caused by atherosclerosis, which is a condition that occurs when the arteries are narrowed due to the buildup of atherosclerotic plaque. Percutaneous transluminal coronary angioplasty (PTCA) is frequently performed to open blocked coronary arteries caused by CAD. However, restenosis (arterial re-narrowing) after PTCA was a major limitation and required second revascularization procedure in 30-40% of the patients. Implantation of metal stents reopened the narrowed arteries and provided scaffolding which eliminates vessel recoil and negative remodeling (vessel shrinkage). However, in-stent restenosis because of neo-intima (new tissue) formation remains a significant problem. Drug-eluting stents, which release anti-proliferative drugs for localized delivery, are a major advancement in the evolution of stents. However, in some instances, there has been late stent thrombosis in patients having drug eluting stents.

As shown in FIG. 1, most drug eluting stents 10 release anti-proliferative drugs in abluminal (towards vessel wall 12 as shown by arrows A) as well as luminal (towards lumen 18 as shown by arrows B) directions. While the abluminal release of anti-proliferative drugs toward the vessel wall 12 is highly beneficial in controlling the growth of smooth muscle cells 14 and thereby inhibiting neointimal hyperplasia, the luminal release of such drugs into the lumen 18 impedes re-endothelialization (the re-growth of the endothelial cell lining 16 on luminal stent surfaces). The re-endothelialization of luminal stent surfaces is of paramount importance because the complete endothelial cell lining prevents the adhesion and aggregation of blood platelets and thereby inhibits late stent thrombosis. Hence, there is a need to provide for directional drug elution in order to promote the growth of endothelial cells 16 on the luminal stent surfaces while inhibiting neointimal hyperplasia.

### BRIEF SUMMARY OF THE INVENTION

Implantable medical devices may directionally elute a first therapeutic agent that promotes the growth of endothelial cells and a second therapeutic agent that inhibits the growth of smooth muscle cells. In some embodiments, implantable medical devices may elute a first therapeutic agent such as an anti-proliferative drug from an abluminal side of the implantable medical device and a second therapeutic agent such as an endothelialization agent from a luminal side of the implantable medical device. In some embodiments, an implantable medical device may be a stent or a vascular graft.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a schematic illustration of a known implantable medical device using known technologies.

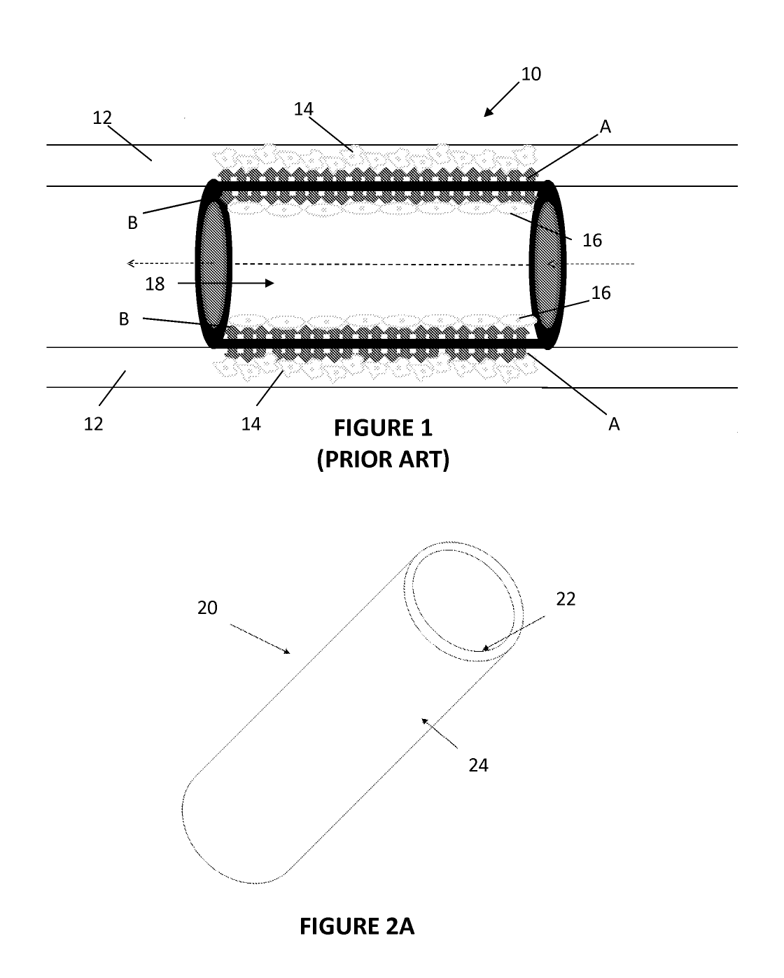

FIG. 2A is a schematic illustration of an implantable medical device in accordance with embodiments of the disclosure.

FIG. 2B is a schematic illustration of a portion of a stent strut in accordance with embodiments of the disclosure.

FIG. 2C is a schematic illustration of an implantable stent in accordance with certain embodiments of the disclosure.

FIG. 2D is a schematic illustration of a method of coating a stent in accordance with certain embodiments of the disclosure.

FIGS. 3A-3E are graphical representations of FTIR data as described in Examples One, Two, Three, and Five.

FIG. 4 provides SEM images of abluminal stent surfaces prior to coating as described in Example Two.

FIG. 5 provides SEM images of luminal stent surfaces prior to coating as described in Example Two.

FIG. 6 provides SEM images of abluminal stent surfaces after coating with paclitaxel as described in Example Two.

FIG. 7 provides SEM images of luminal stent surfaces after coating with DETA NONO as described in Example Three.

FIGS. 8A-8E provide optical profilometry characterizations of coated surfaces as described in Example Four.

FIGS. 9A-9D provide SEM images of co-coated stent surfaces after coating with paclitaxel and DETA NONOate as described in Example Five.

FIGS. 10A-10C provide optical profilometry characterizations of coated surfaces as described in Example Five.

FIGS. 11A-11G provide SEM images of stent surfaces after the stents have been expanded as described in Example Six.

FIGS. 12A-12J provide contact angle images of stent surfaces as described in Example Seven.

FIGS. 13A-13F are graphical representations of therapeutic agent elution as described in Example Eight.

FIG. 14 is a schematic illustration of an implantable stent in accordance with certain embodiments of the disclosure.

FIGS. 15A-15D are SEM images of Co—Cr alloy surfaces coated with PEO and, in some cases, the PEO coating contains various concentrations of paclitaxel.

FIG. 16A provides an SEM image of a Co—Cr alloy surface coated with heparin, while FIG. 16B provides an SEM image of a Co—Cr alloy surface coated with DETA NONOate incorporated heparin.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

An implantable medical device may directionally elute a first therapeutic agent from a first surface and may directionally elute a second therapeutic agent from a second surface. The first therapeutic agent and the second therapeutic agent may be the same or different. In some instances, the first therapeutic agent is eluted in a first direction for a first purpose or function, and the second therapeutic agent is eluted in a second direction for a second purpose or function. In some embodiments, an implantable medical device may directionally elute a first therapeutic agent that promotes the growth of endothelial cells and a second therapeutic agent that inhibits the growth of smooth muscle cells. In some embodiments, implantable medical devices may elute a first therapeutic agent such as an anti-proliferative drug from an abluminal side of the implantable medical device and a second therapeutic agent such as an endothelialization agent from a luminal side of the implantable medical device.

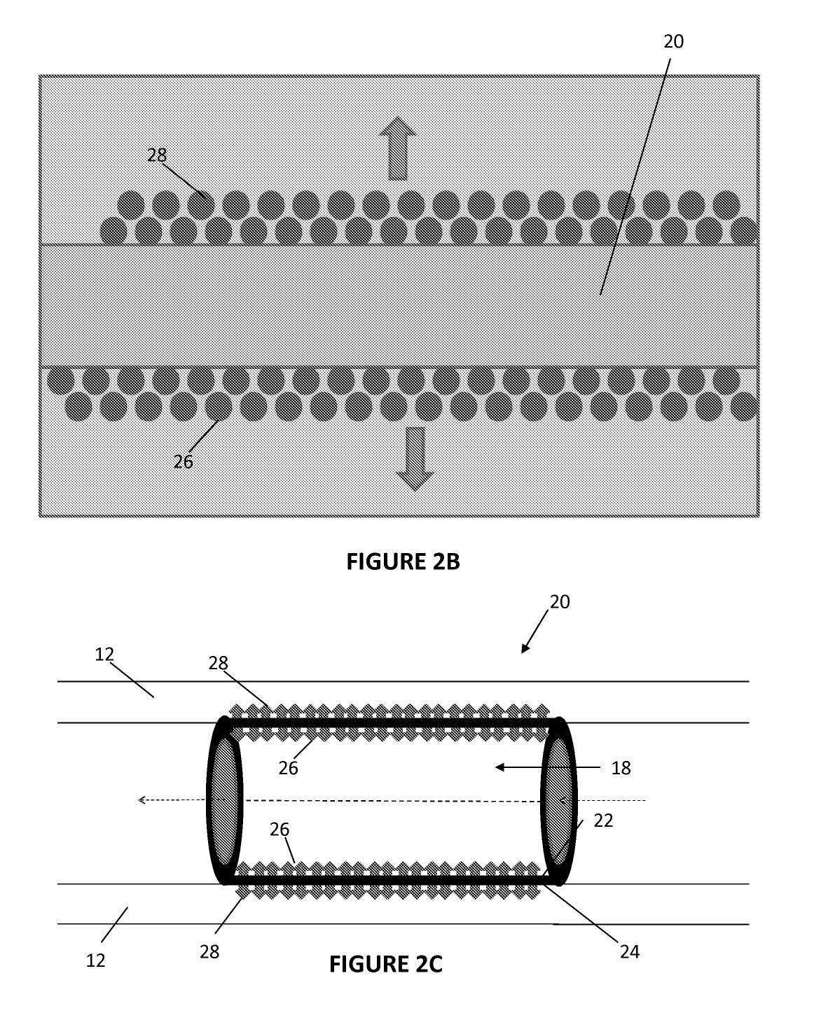

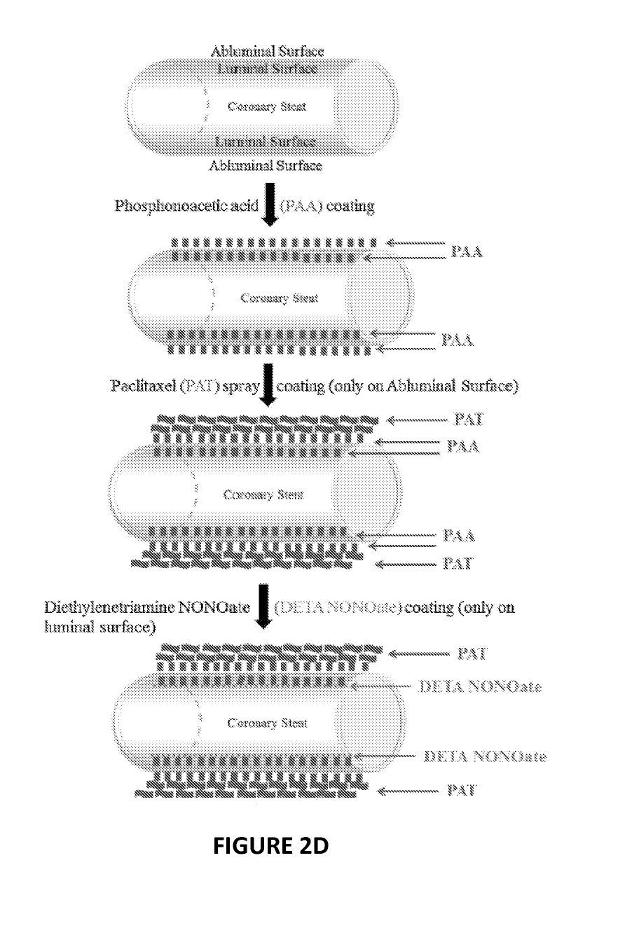

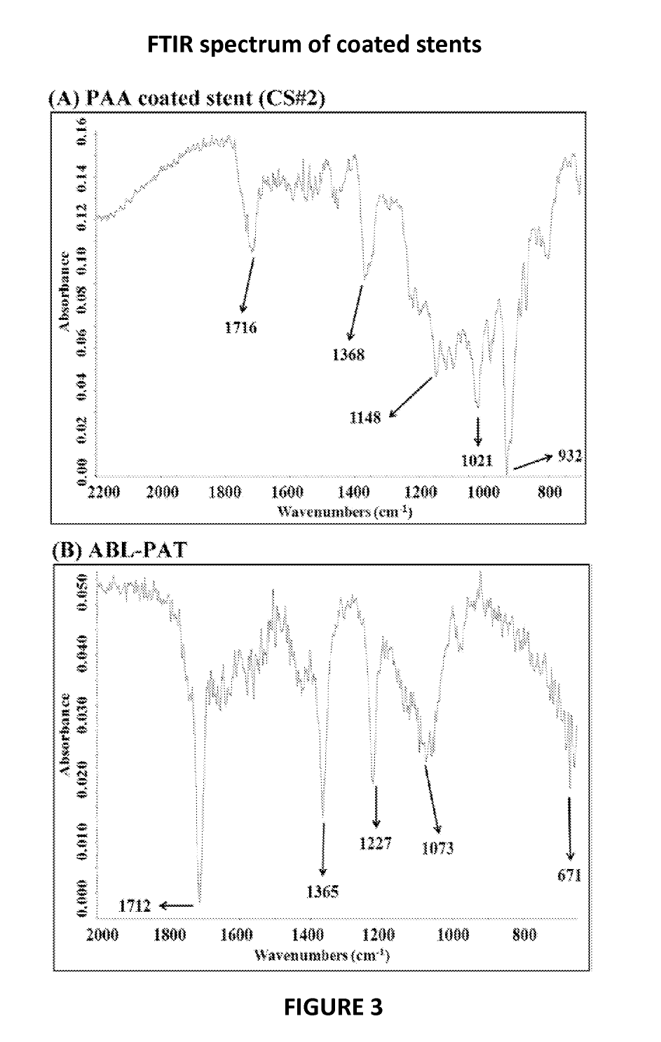

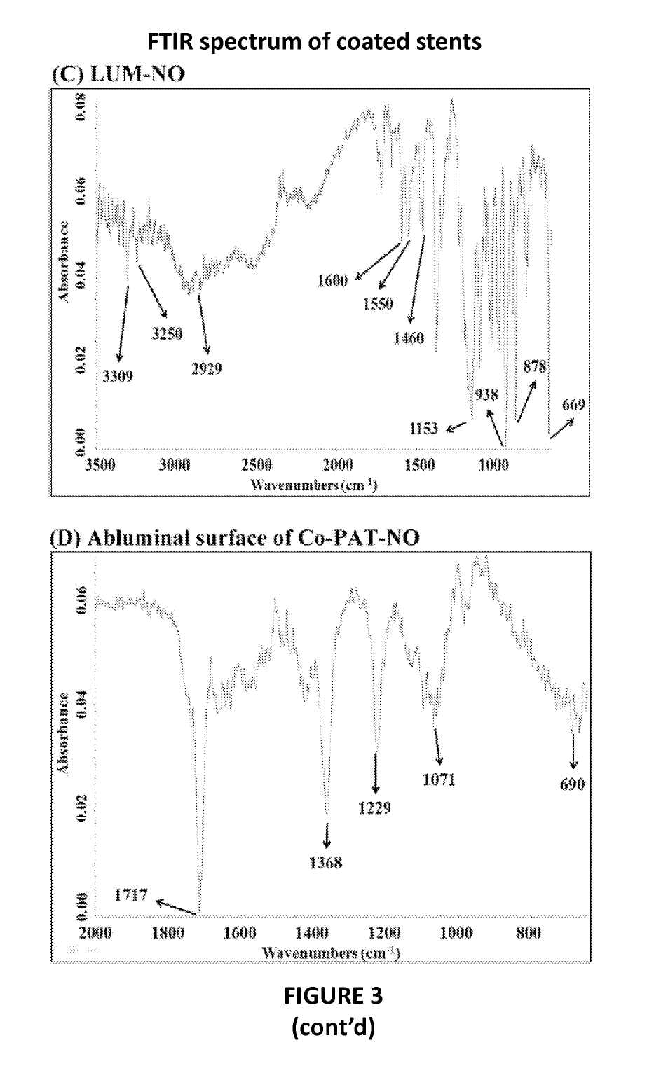

FIGS. 2A, 2B, and 2C are schematic illustrations of an implantable medical device 20. The implantable medical device 20 generally includes an inner surface 22 and an outer surface 24. The implantable medical device 20 may be formed of or otherwise include a variety of metallic, polymeric or ceramic substrates. It will be appreciated that the implantable medical device 20 schematically represents a variety of different implantable medical devices or portions thereof. Illustrative but non-limiting examples of implantable medical devices 20 include stents and vascular grafts. In general, any implantable device having an inner surface and an outer surface is contemplated herein.

In some embodiments, the implantable medical device 20 may be a stent. Stents may be formed of metallic materials, polymeric materials and ceramic materials. Illustrative but non-limiting examples of metallic materials include stainless steel, tantalum and tantalum alloys, titanium and titanium alloys including NITINOL, platinum-iridium alloys, magnesium and magnesium alloys and cobalt-chromium alloys.

In some embodiments, at least one of the inner surface 22 and the outer surface 24 may be processed to include functional groups that bond to at least one of the inner surface 22 and the outer surface 24. Therapeutic agents may then be bonded to the functional groups. In some embodiments, the inner surface 22 and the outer surface 24 may be treated to include the same functional group. As best shown in FIGS. 2B and 2C, a first therapeutic agent 26 may be bonded to the functional groups disposed on the inner surface 22 and a second therapeutic agent 28 may be bonded to the functional groups disposed on the outer surface 24. In some embodiments, the first 26 and second 28 therapeutic agents may be different, and may be selected for different purposes and needs.

Examples of suitable functional groups include but are not limited to hydroxyl groups (—OH), carboxylic acid groups (—COOH) and amine groups (—NH₂). Antiproliferative drugs such as paclitaxel and nitric oxide donor drugs such as DETA NONOate may form hydrogen or covalent bonds with these functional groups. It will be appreciated that there are a variety of ways to add these functional groups to the inner surface 22 and the outer surface 24, depending on the chemical makeup of the implantable medical device 20.

In some embodiments, the implantable medical device 20, particularly if formed of a metal, may be treated using phosphonoacetic acid, which has the chemical structure shown below:

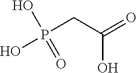

In some embodiments, an implantable medical device 20 may be treated by immersing the device in an aqueous solution of phosphonoacetic acid, followed by allowing the treated device to dry at an elevated temperature.

Once the phosphonoacetic acid has been bonded to the implantable medical device 20, one or more therapeutic agents may subsequently be bonded to the bound phosphonoacetic acid. In some embodiments, a first therapeutic agent 26 such as an endothelialization promotion agent may be applied to the inner surface 22, and a second therapeutic agent 18 such as an antiproliferative agent may be applied to the outer surface 24.

Illustrative but non-limiting examples of antiproliferative agents include Sirolimus, Everolimus, Zotarolimus, Tacrolimus, Umirolimus, Pimecrolimus, Dexamethasone, Paclitaxel and aspirin. Illustrative but non-limiting examples of endothelialization promotion agents include L-ascorbic acid (vitamin C) and sources of nitric oxide. Nitric oxide sources include compounds that naturally elute or evolve nitric oxide. Examples include diethylenetriamine/nitric acid adducts such as DETA NONOate, which has the chemical structure shown below:

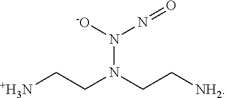

In some embodiments, the implantable medical device 20 is a stent. Once the stent has been treated with phosphonoacetic acid, the inner surface 22 may be coated with a nitric oxide donor and the outer surface 24 may be treated with paclitaxel. It will be appreciated that under physiological conditions, the bound phosphonoacetic acid carries negatively charged —COO— groups that will form electrostatic interactions with positively charged —NH₃₊ groups present within the nitric oxide donor. Paclitaxel includes —OH groups and thus will form hydrogen bonds with —COOH groups of the bound phosphonoacetic acid.

There are multiple ways to coat the inner surface 22 with a first therapeutic agent 26 such as a nitric oxide donor and to coat the outer surface 24 with a second therapeutic agent 28 such as paclitaxel. In some embodiments, the implantable medical device 20 is processed such that the inner surface 22 includes very little paclitaxel and the outer surface 24 includes very little nitric oxide donor.

In one example, the inner 22 and outer 24 surfaces of a stent 20 may be contacted with phosphonoacetic acid. The outer surface 24 of the stent 20 may then be masked prior to spraying a first therapeutic agent 26 such as a nitric oxide donor onto the inner surface 22 of the stent 20. In some embodiments, masking the outer surface 24 will result in an outer surface 24 that is at least substantially free of the first therapeutic agent 26 (such as a nitric oxide donor). A second therapeutic agent 28, such as paclitaxel, may then be sprayed onto the outer surface 24 of the stent 20, resulting in an inner surface 22 that is at least substantially free of the second therapeutic agent 28 (such as paclitaxel).

In another example, the inner 22 and outer 24 surfaces of a stent 20 may be contacted with phosphonoacetic acid. The outer surface 24 of the stent 20 may be sprayed with a second therapeutic agent 28, such as paclitaxel. A mandrel (not shown) may be coated with a first therapeutic agent 26, such as a nitric oxide donor. The stent 20 may be placed on the mandrel in order to transfer the first therapeutic agent 26 (such as a nitric oxide donor) from the mandrel to the inner surface 22 of the stent 20.

In another example, a polymer containing a second therapeutic agent 28 (such as a paclitaxel-containing polymer) may be coated onto the outer surface 24 of the stent 20. And a polymer containing a first therapeutic agent 26 (such as a nitric oxide donor-containing polymer) may be coated onto the inner surface 22 of the stent 20. It will be appreciated that either coating may be done first, i.e., the outer surface 24 may be coated first, followed by coating the inner surface 22, or the inner surface 22 may be coated before coating the outer surface 24.

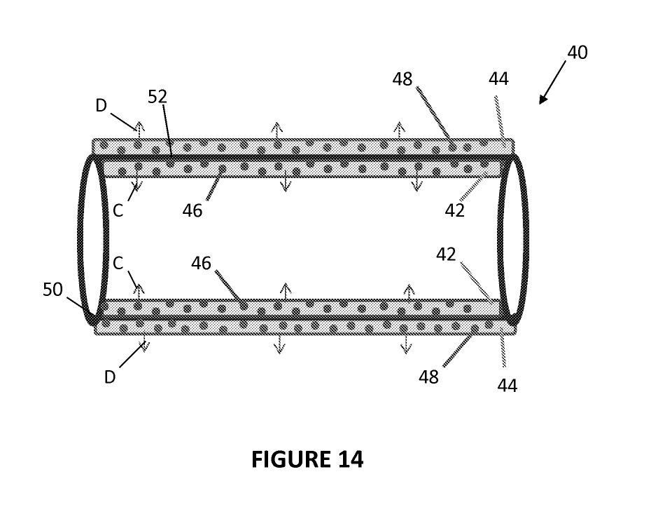

FIG. 14 depicts an exemplary stent 40 having a first polymer 42 coated on the inner surface 50 of the stent 40 and a second polymer 44 coated on the outer surface 52. The first polymer 42 contains the first therapeutic agent 46, which is embedded or otherwise contained within the first polymer 42. In one exemplary embodiment, the first polymer 42 is heparin 42, and the first therapeutic agent 46 is DETA NONOate. The second polymer 44 contains the second therapeutic agent 48, which is embedded or otherwise contained within the second polymer 44. In one exemplary implementation, the second polymer 44 is polyethylene oxide (“PEO”) 44, and the second therapeutic agent 48 is paclitaxel.

In accordance with one embodiment, the polyethylene oxide 44 coated on the outer surface 52 can control the delivery or elution of the second therapeutic agent 48, as shown by arrows D. Further, the polyethylene oxide coating 44 has characteristics that provide resistance to smooth muscle cell attachment and growth on the stent 40. More specifically, polyethylene oxide resists protein adsorption and thus can resist or prevent cell adhesion. Thus, like the second therapeutic agent 48, the PEO coating 44 can help to prevent attachment and growth of smooth muscle cells. As a result, the PEO coating 44 can work in combination with the second therapeutic agent 48 to resist attachment and growth of smooth muscle cells on the outer surface 52 of the stent 40. Further, when all of the second therapeutic agent 48 has eluted from or been released from the PEO coating 44, the PEO coating 44 itself can still resist attachment and growth of smooth muscle cells.

In one implementation, the heparin coating 42 coated in the inner surface 50 can control delivery or elution of the first therapeutic agent 46, as shown by arrows C. Further, the heparin coating 42 has anti-thrombogenic properties. Thus, like the first therapeutic agent 46, the heparin coating 42 can help to inhibit late stent thrombosis. As a result, the heparin coating 42 can work in combination with the first therapeutic agent 46 to inhibit late stent thrombosis. Further, when all of the first therapeutic agent 46 has eluted from or been released from the heparin coating 42, the heparin coating 42 itself can still inhibit late stent thrombosis.

FIG. 2B is a schematic illustration of a stent strut 20 (a portion of a stent 20) according to a further embodiment, and is essentially a close-up of a portion of the stent in FIG. 2C. It can be seen that a first therapeutic agent 26 (for example, a nitric oxide donor such as DETA NONOate) is disposed on the luminal side 22 of the stent 20 and a second therapeutic agent 28 (for example, an antiproliferative drug such as paclitaxel) is disposed on the abluminal side 24 of the stent strut 20. Accordingly, in this particular example, the exemplary paclitaxel 28 is positioned proximate to the vessel wall 12 while the exemplary nitric oxide donor 26 is positioned proximate the lumen 18 of the stent 20.

FIG. 2D depicts another implementation relating to a method of coating a stent. First, the outer surface is coated with phosphonoacetic acid, and then with a spray coating of paclitaxel (only on the outer surface). Then diethylenetriamine NONOate is coated only on the inner surface.

Accordingly, a stent 20 treated in this manner may be used in a method of controlling neointimal hyperplasia along an outer surface 24 of the stent 20 and encouraging growth of endothelial cells along an inner surface 22 of the stent 20. A stent 20 having a first therapeutic agent 26 on an inner surface 22 of the stent 20 and a second therapeutic agent 28 on an outer surface 24 of the stent 20 may be implanted within a patient's vasculature. The first therapeutic agent 26 may be eluted from the inner surface 22 of the stent 20. The second therapeutic agent 28 may be eluted from the outer surface 24 of the stent 20.

In some embodiments, the first therapeutic agent 26 may be eluted only from the inner surface 22 of the stent 20 and the second therapeutic agent 28 may be eluted only from the outer surface 24 of the stent 20. In some embodiments, the first therapeutic agent 26 may be an endothelialization growth agent such as a nitric oxide donor, while the second therapeutic agent 28 may be an anti-proliferative agent such as paclitaxel.

### EXAMPLES

A variety of experiments were carried out to demonstrate directional elution of therapeutic agents from an implantable medical device such as a stent.

### Example One

In Example 1, Co—Cr alloy stents were immersed in ImM solution of phosphonoacetic acid in de-ionized water (di-H₂0) for 24 hours followed by heating the stents in air at 120° C. for 18 hours. The stents were then cleaned by sonication in di-H₂0 for 1 minute and dried using nitrogen gas. Thus prepared phosphonoacetic acid coated stents were characterized using Fourier transform infrared spectroscopy (FTIR).

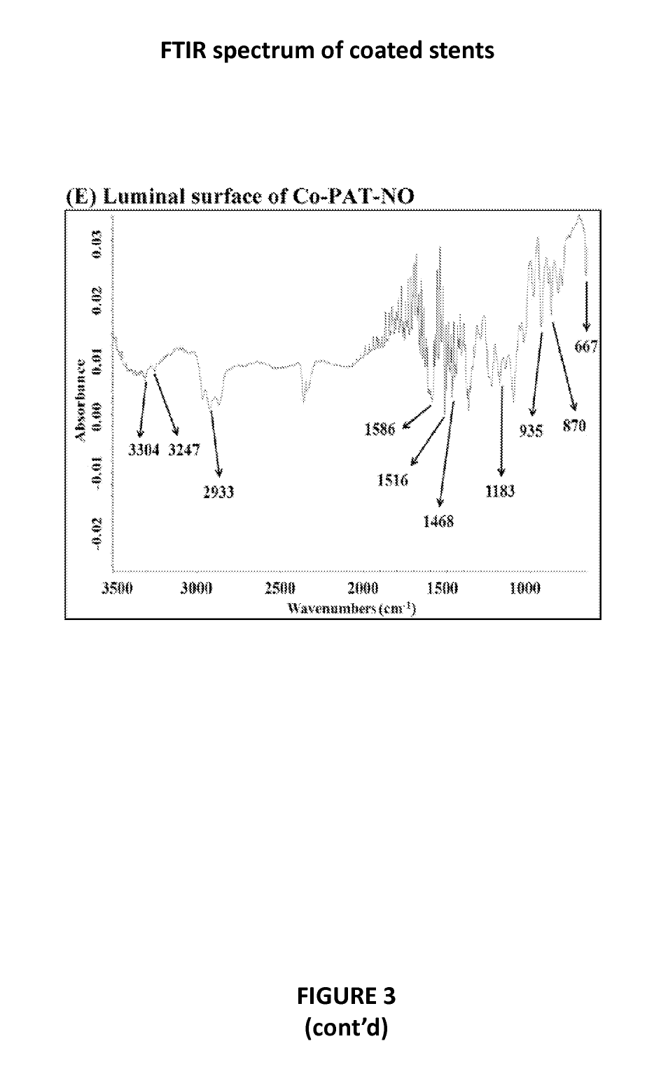

FIG. 3A provides the FTIR results. The FTIR spectrum of phosphonoacetic acid coated stents showed peak positions at 932, 1021, 1148, and 1716 cm-¹ which were assigned to P—OH, P—O-Metal, P═O, and C═O functionalities on the stents, respectively. The peak for the P—O-Metal at 1021 cm-¹ shows that the phosphonoacetic acid is covalently bound to Co—Cr alloy stents. The C═O stretch at 1716 cm-¹ shows the presence of —COOH terminal groups on the stent surface. Also, the peaks for P—OH and P═O further confirms the presence of phosphonoacetic acid on stents. Thus, the FTIR confirms the phosphonoacetic acid coating on Co—Cr alloy stents.

### Example Two

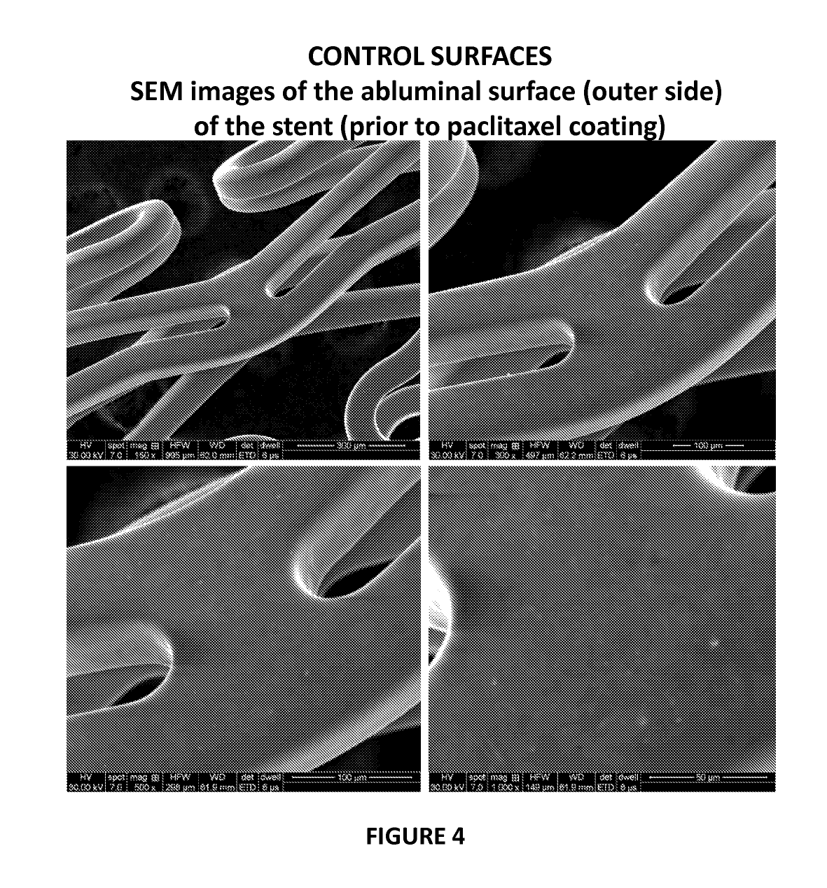

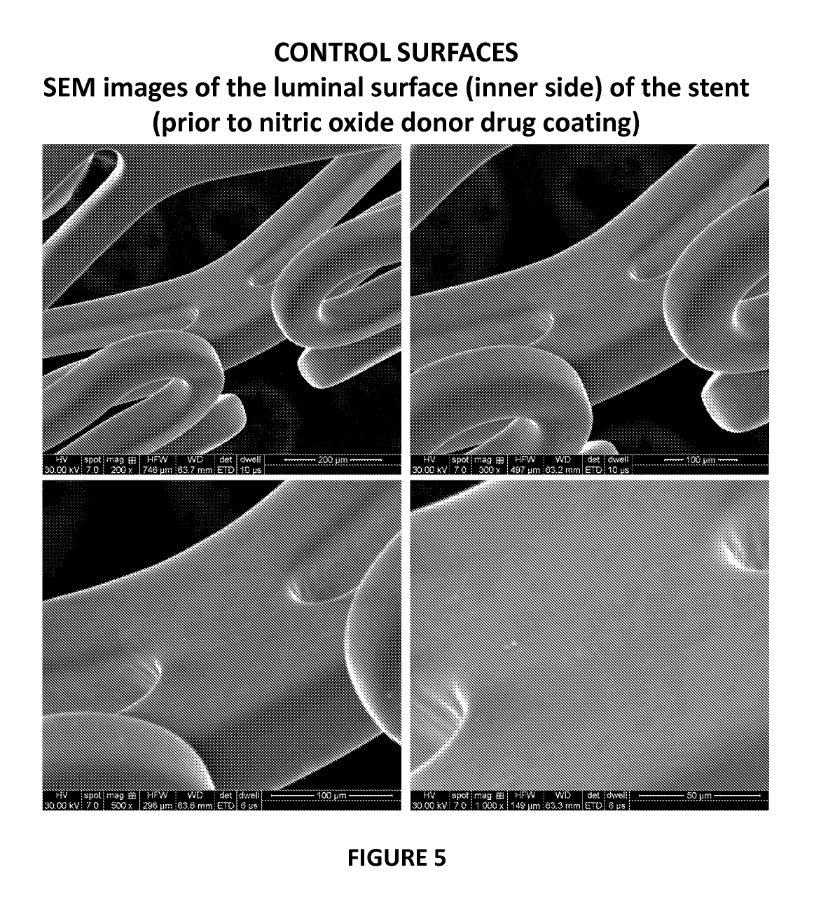

In Example 2, the abluminal surface of a phosphonoacetic acid-treated stent was coated with paclitaxel. FIGS. 4 and 5 show the abluminal and luminal surfaces, respectively, of the Co—Cr alloy stents prior to coating.

The phosphonoacetic coated stents were placed on a mandrel in such a way that the luminal surface of the stents was in close touch (tight contact) with the mandrel. A solution of paclitaxel was prepared in 75% ethanol and 25% DMSO. Thus prepared paclitaxel solution was sprayed on the abluminal surfaces of the stent. A tight contact was maintained between the luminal stent surface and the mandrel to prevent any paclitaxel moving into the luminal surface of the stent. In addition, once the spray coating was finished, the stent (coated with paclitaxel on the abluminal surface) was taken out and luminally cleaned to make sure there is no paclitaxel present on the luminal surface of the stent.

This exclusive luminal surface cleaning was carried out by the following procedure. A mandrel was immersed in ethanol and the stent (coated with paclitaxel on the abluminal surface) was placed on the ethanol immersed mandrel. The stent was then moved back and forth to remove any paclitaxel present on the luminal surface of the stent. Ethanol was used in this luminal surface cleaning procedure since ethanol is an excellent solvent for paclitaxel. Thus, the paclitaxel was coated on the abluminal surface of the stent without coating it on the luminal surface of the stent. The stent surfaces were characterized before and after coating with paclitaxel on the abluminal surface.

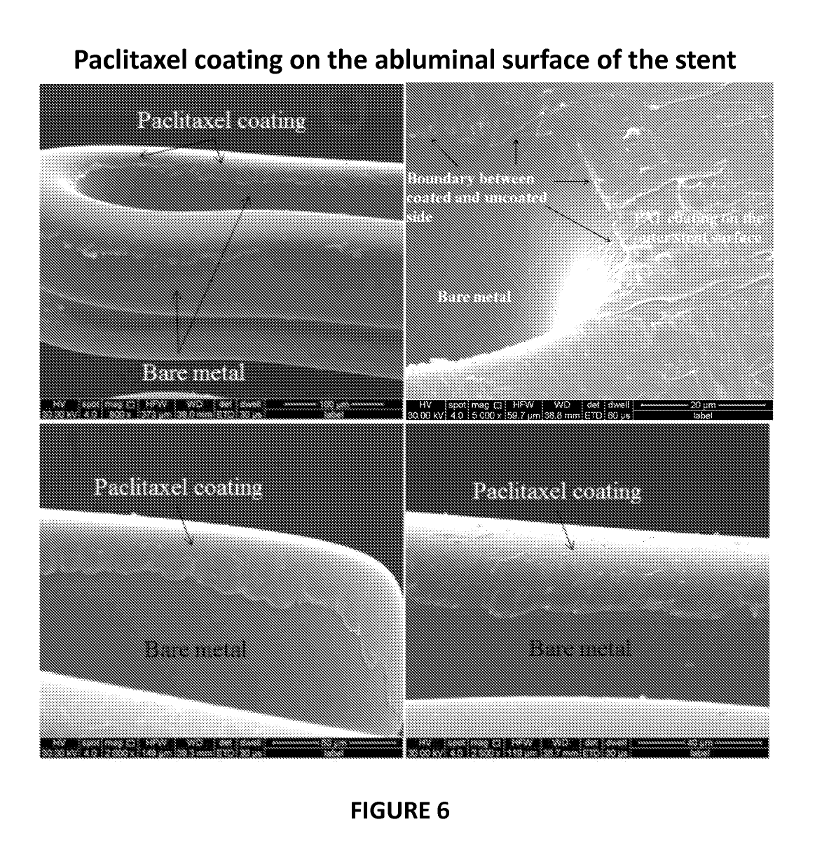

FIG. 6 shows the SEM images of the abluminal surface of the stent after the deposition of paclitaxel. The paclitaxel formed a film on the abluminal surfaces of the phosphonoacetic acid coated stents. Paclitaxel was coated on phosphonoacetic acid functionalized stent surfaces by extensive hydrogen bonding interactions between the —OH groups of drug and —COOH groups of phosphonoacetic acid. The portions labeled as “bare metal” are free of paclitaxel but have a phosphonoacetic acid coating. Comparing FIG. 4 to FIG. 6 illustrates how the paclitaxel is coated only on the abluminal surfaces.

FIG. 3B provides the FTIR results. The FTIR spectrum of paclitaxel deposited on the abluminal surface of the stent showed peak positions at 671, 1073, 1227, 1365, and 1712 cm-¹. These peak positions are in agreement with the literature for the paclitaxel coating. Thus, the FTIR confirms the successful deposit of paclitaxel on the phosphonoacetic acid coating on Co—Cr alloy stents.

### Example Three

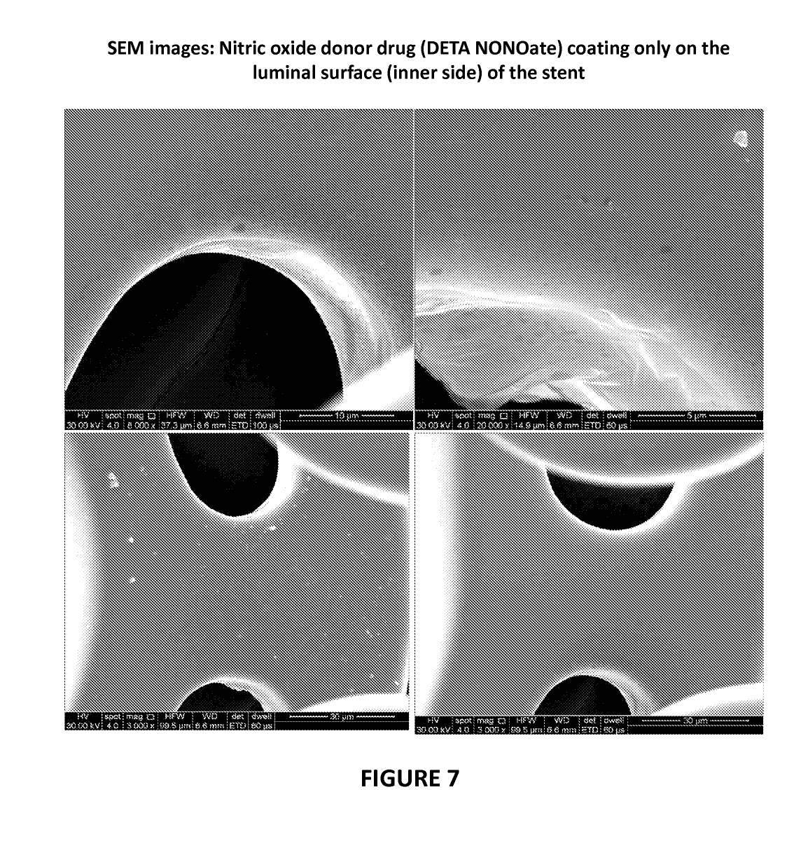

In Example 3, the luminal surface of a Co—Cr stent was coated with a nitric oxide donor drug. A 5 mM solution of DETA NONOate (diethylenetriamine NONOate) was prepared in di-H₂0. A clean mandrel was placed in a 3 mL of DETA solution for 30 minutes. The mandrel was removed from the solution and the stent was placed onto the mandrel for 5 minutes to allow transferring the DETA NONOate from the mandrel to the luminal surface of the stent. The stent was then removed from the mandrel and allowed to dry in air for 15 minutes. Thus, the nitric oxide donor drug, DETA NONOate, was coated only on the luminal surfaces of the stent. The stent surfaces were characterized using scanning electron microscopy, as shown in FIG. 7.

FIG. 7 shows the SEM images of the luminal surfaces of the stent after the deposition of DETA NONOate. DETA NONOate formed a molecular coating on the luminal surfaces of the phosphonoacetic acid coated stent. The phosphonoacetic acid coating carry negatively charged groups (—COO—) under physiological conditions while the DETA/NO adduct has positive charge (NH₃₊) groups. Thus, DETA/NO was coated on phophonoacetic acid functionalized stents by electrostatic attractions. Comparing FIG. 5 to FIG. 7 illustrates how the DETA NONOate is coated only on the luminal surfaces.

FIG. 3C provides the FTIR results. The FTIR spectrum of DETA NONOate deposited on the luminal surface of the stent showed peak positions that are fingerprint regions at 669, 878, 938, and 1153 cm-¹. The peak for the scissoring vibration of —CH₂ groups was observed at 1460 cm-¹. The peaks for the N═O and N—O stretches were observed at 1550 and 1600 cm-¹, respectively. A broad peak for the NH₃⁺ was observed at 2929 cm-¹. Also, the symmetric and asymmetric stretches of N—H groups were observed at 3250 cm-¹ and 3309 cm-¹, respectively. Thus, the FTIR confirms the successful coating of DETA NONOate on the luminal surface.

### Example Four

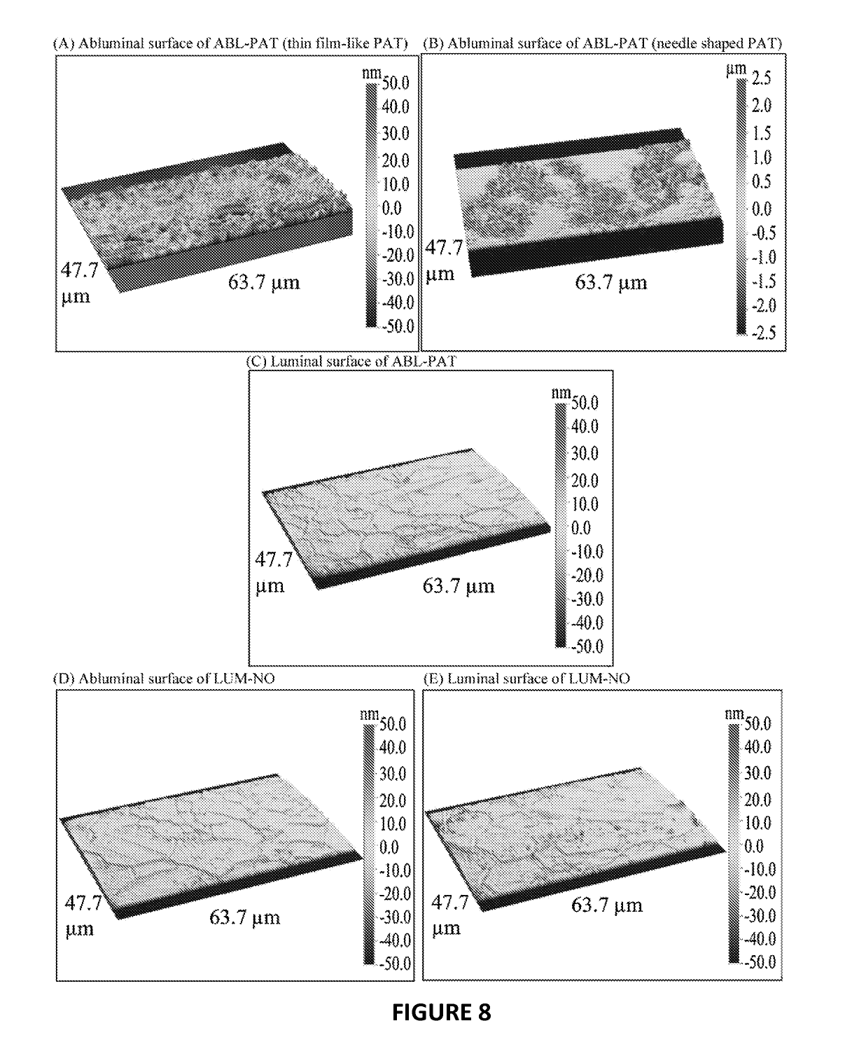

In Example 4, the drug coated stents of Example 3 underwent optical profilometry characterization. The results are shown in FIG. 8. FIGS. 8A and 8B show the thin film-like morphology and needle-shaped morphology of paclitaxel on the abluminal surfaces of the stent, respectively. In both images, the underlying metal microstructure is not visible, which suggested that the paclitaxel was uniformly coated on the abluminal stent surfaces. As expected, a significant increase in the surface roughness value was observed for the abluminal surface of the stent when compared to that of the abluminal surfaces of control surfaces (without a therapeutic agent deposited).

As shown in FIG. 8C, the topography image of the luminal surface of the stent showed the microstructural grain features. Also, no significant increase in the surface roughness value was observed for the luminal surface when compared to that of the luminal surfaces of stents with no therapeutic agent coating. These results strongly suggest that the paclitaxel was not present on the luminal stent surface.

FIGS. 8D and 8E show the topography images of the abluminal and luminal surfaces of the stent coated on the luminal surface with DETA NONOate. In agreement with the SEM images discussed above, no significant difference in the surface topography was observed between an uncoated stent and the stent coated with DETA NONOate on the luminal surface. This suggests that the DETA NONOate was deposited as a molecular coating which followed the contour of microstructural grain features of the stent surfaces.

### Example Five

In Example 5, a phosphonoacetic acid-treated stent was co-coated with paclitaxel and DETA NONOate. That is, the stent was first spray-coated with paclitaxel only on the abluminal surface as described in Example 2, and then the stent was coated with DETA NONOate on the luminal surface as described in Example 3.

FIGS. 3D and 3E provides the FTIR results. More specifically, FIG. 3D provides the FTIR spectrum for the abluminal surface of the co-coated stent, while FIG. 3E provides the FTIR spectrum for the luminal surface of the co-coated stent. The IR peaks observed show the presence of paclitaxel and DETA NONOate on the abluminal and luminal surfaces of the stent, respectively. The IR peak positions for the paclitaxel on the abluminal surface and DETA NONOate on the luminal surface are in agreement with those of paclitaxel and DETA NONOate as provided in the above examples relating to the other stents. These results show the successful co-coating of paclitaxel and DETA NONOate on the abluminal and luminal surfaces of the stent, respectively.

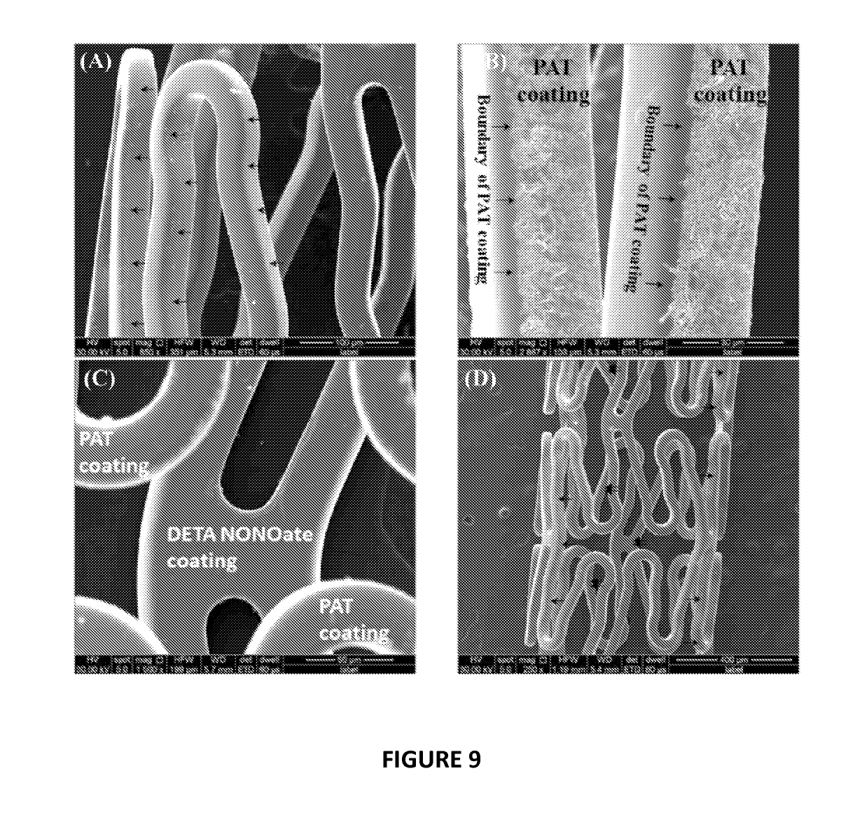

FIGS. 9A-9D show the SEM images of the co-coated stent after the deposition of paclitaxel and DETA NONOate. The coating of paclitaxel on the abluminal stent surfaces as thin film-like structure and needle-shaped crystals are shown in FIGS. 9A and 9B, respectively. The arrows provided in these images show the boundary of PAT coating to confirm that the drug coating did not extend up to the luminal stent surface. FIG. 9C shows the DETA NONOate coated luminal surface of the co-coated stent. A low magnification (250×) image of the stent was provided in FIG. 9D to show that the drug coating was uniformly distributed on the stent surface. In this image, a single arrow indicates the paclitaxel coating on the abluminal surface while a double arrow indicates the DETA NONOate coated luminal surface. Thus, the images confirm that the morphologies and distribution of the drug coating on the co-coated stent are identical to that of the single-coated stents described in the examples above.

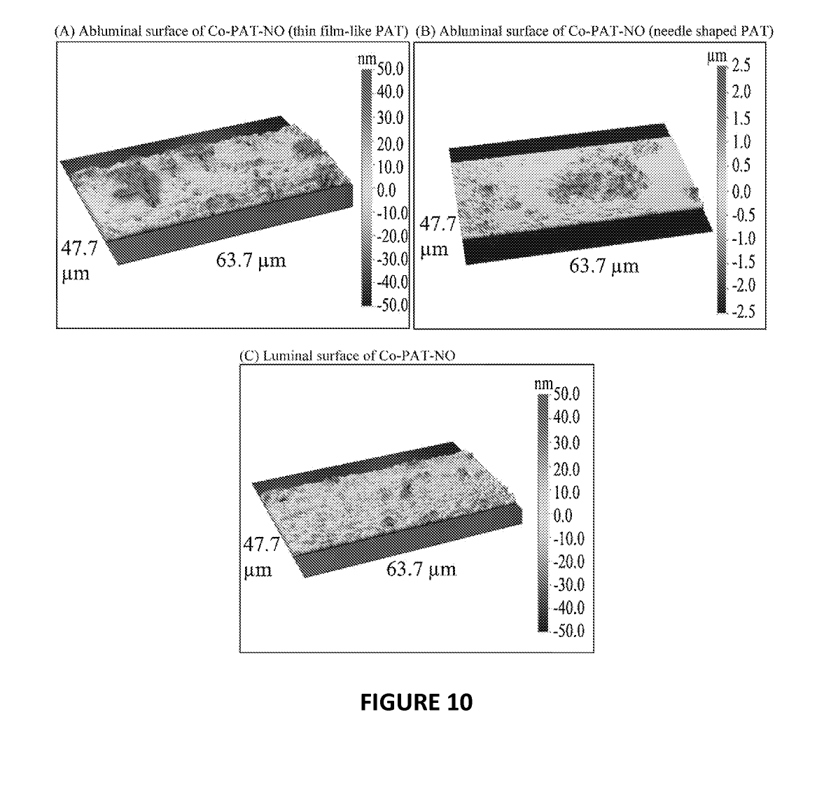

As shown in FIG. 10, the co-coated stent also underwent optical profilometry characterization. The morphologies of the therapeutic agents (including the thin film-like paclitaxel in FIG. 10A, the needle-shaped paclitaxel crystals in FIG. 10B, and the DETA NONOate molecular coating in FIG. 10C) observed in the co-coated stent were consistent with that of single drug coated stents as described above. The luminal surfaces of the DETA NONOate coated stent in FIG. 8E and this co-coated stent in FIG. 10C appear to be different because of differences in preparation. That is, for the co-coated stent, after paclitaxel coating on the abluminal surface, the luminal surface alone was cleaned using an ethanol wetted mandrel. In contrast, no such procedure was performed with respect to the DETA NONOate coated stent in FIG. 8E, because there was no paclitaxel coating on the abluminal surface of that stent. Hence, the luminal surface of the co-coated stent appears rougher when compared to that of the DETA NONOate coated stent in FIG. 8E.

### Example Six

In Example 6, the control stent (described in Example 1 above) and the co-coated stent (described in Example 5 above) were expanded, just as they would be expanded during use. That is, each stent was expanded using a standard angioplasty balloon catheter to examine the impact of expansion on the coatings/deposits.

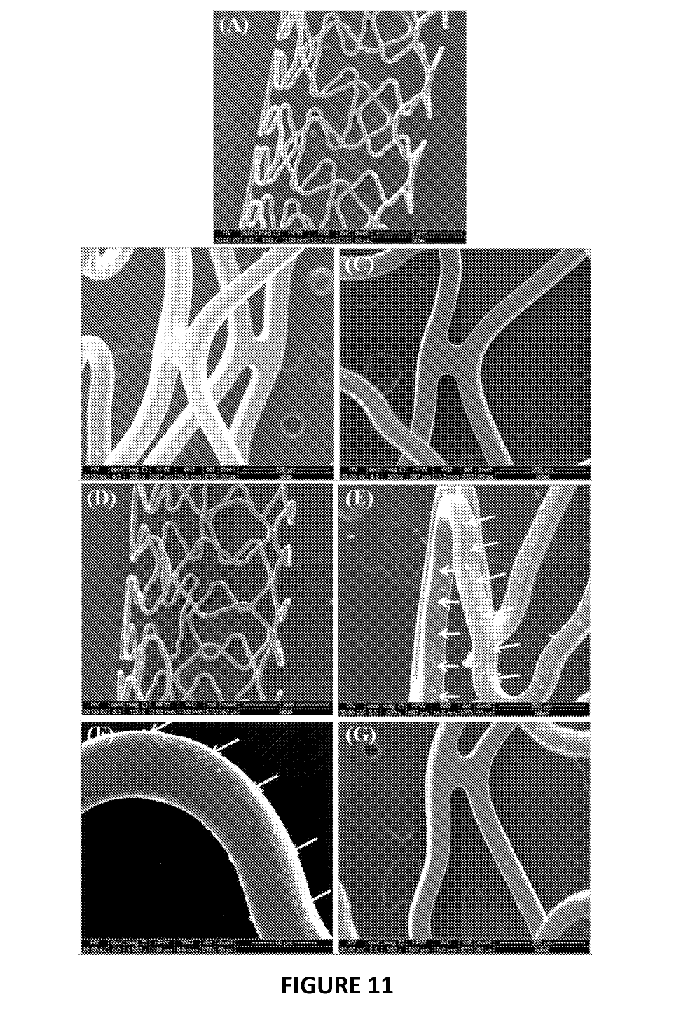

FIGS. 11A-11G show the SEM images of the expanded stents. Both low (100×) and high magnification (500× and 1500×) SEM images were captured to study the integrity of the coatings after expansion. FIG. 11A shows the low magnification image of the expanded control stent of Example 1, while FIGS. 11B and 11C show the high magnification images of the abluminal and luminal surfaces of the expanded control stent, respectively. FIG. 11D shows the low magnification image of the co-coated stent of Example 5. FIGS. 11E and 11F show the high magnification images of the abluminal surfaces of the co-coated stent. The arrows in these figures show the boundary of paclitaxel coating on the abluminal surface. FIG. 11G shows the high magnification image of the luminal surface of the co-coated stent.

No delamination or cracking of the drug coatings was observed on the co-coated stent surfaces. As a result, these results demonstrate that the integrity of the co-coating was maintained during the stent expansion procedure.

### Example Seven

In Example 7, the contact angles were examined for each of the stents discussed in the above examples.

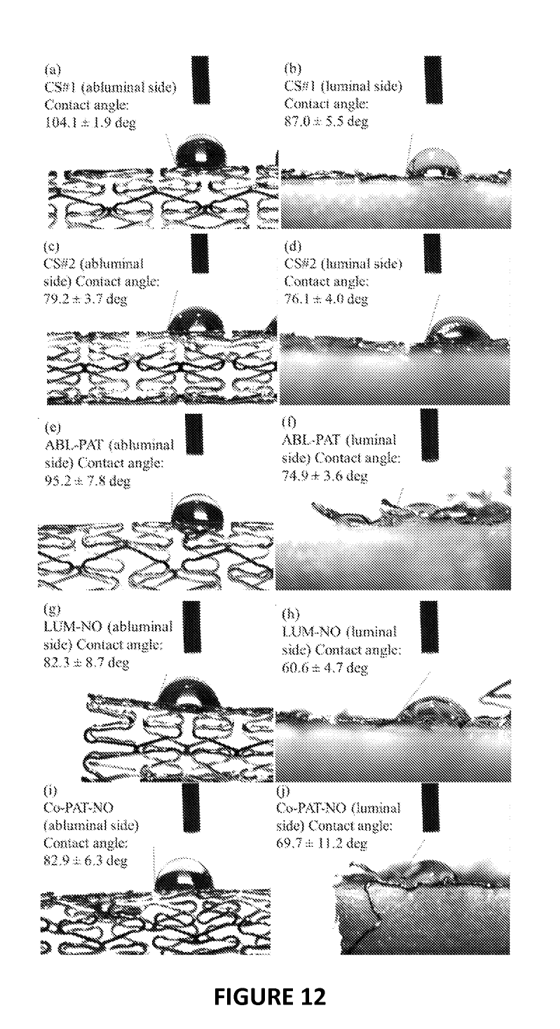

FIGS. 12A-12J show images of the contact angles obtained for the abluminal and luminal surfaces of the stents. As shown in FIGS. 12A and 12B, the contact angles of the abluminal and luminal surfaces of an uncoated control stent were measured as 104.1±1.9° and 87±5.5°, respectively. As shown in FIGS. 12C and 12D, after coating with phosphonoacetic acid as described in Example 1, the contact angles significantly decreased to 79.2±3.7° and 76.1±4° for the abluminal and luminal stent surfaces, respectively. A decrease in the contact angle after phosphonoacetic acid coating was expected since the phosphonoacetic acid contains hydrophilic —COOH terminal groups (see paragraph 25 above). As shown in FIG. 12E, for the stent of Example 2 (coated on the abluminal surface with paclitaxel), an increase in the contact angle (95.2±7.8°) was observed for the abluminal surface of the stent. Although paclitaxel is primarily a hydrophobic drug containing several aromatic rings and —CH₃ functional groups, few hydrophilic functional groups such as —OH, C═O, —COO, and —NH are present in its chemical structure. Hence, the contact angle of paclitaxel can vary from 80° to 100° depending on the orientation of different functional groups and the type of morphology that the paclitaxel crystals can form on a material surface. No significant difference in the contact angle was observed for the luminal surface of the Example 2 stent (74.9±3.6°) depicted in FIG. 12F in comparison to the luminal surface of the Example 1 stent (76.1±4°) depicted in FIG. 12D, which suggested that the paclitaxel was not present on the luminal stent surface, as expected. In contrast, the luminal surface of the Example 3 stent (coated on the luminal surface with DETA NONOate), showing a contact angle of 60.6±4.7° (as shown in FIG. 12H), was more hydrophilic than that of the luminal surface of the Example 1 stent depicted in FIG. 12D. This is because the DETA NONOate is primarily a hydrophilic drug containing several hydrophilic functional groups (—NH₂, N═O, —NH₃⁺, and —NO⁻) in its chemical structure. No significant difference in the contact angle was observed between the abluminal surfaces of the Example 3 stent (82.3±8.7°) (as shown in FIG. 12G) and the Example 1 stent depicted in FIG. 12C (79.2±3.7°). As shown in FIGS. 121 and 12J, respectively, the contact angles of the abluminal and luminal surfaces of the co-coated stents were measured as 82.9+6.3* and 69.7+11.2*.

In agreement with other characterization techniques, these contact angle values also show the successful deposition of paclitaxel and DETA NONOate on the abluminal and luminal surfaces of the stents.

### Example Eight

In Example 8, the drug coated stents of the above examples underwent drug release studies. The drug coated stents were immersed in 2 mL of PBS/Tween-20 (pH=7.4) and incubated in a circulating water bath (Thermo Scientific, USA) at 37° C. At pre-determined time points (1 hour, 3 hours, 6 hours, 12 hours, and 24 hours, and every day thereafter for up to 14 days, followed by day 21 and day 28), the stent samples were taken out of the PBS/T-20 solution and moved to fresh PBS/T-20 solution. The PBS/T-20 solutions collected at each time point were analyzed for the amount of drug (paclitaxel or nitric oxide) released. The amount of paclitaxel released was determined using high performance liquid chromatography (HPLC). The amount of nitric oxide (NO) released was determined using Griess reagent based nitrate/nitrite colorimetric assay.

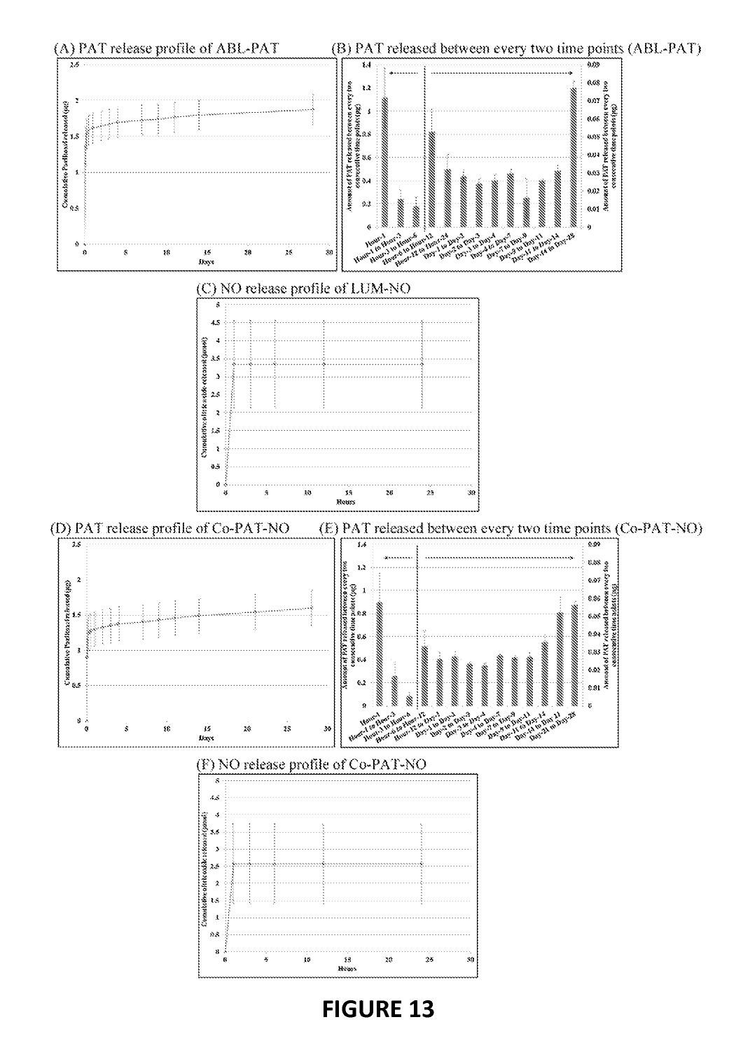

FIG. 13A shows a graphical representation of the in vitro release profile of paclitaxel from the stent of Example 2 (coated on the abluminal surface with paclitaxel). A biphasic release profile with an initial burst followed by a slow and sustained release was observed. FIG. 13B shows the actual amount of paclitaxel released between every two consecutive time points. In this figure, from “Hour-1” to “Hour-3 to Hour-6” were plotted with respect to primary Y-axis while “Hour-6 to Hour-12” to “Day-14 to Day-28” were plotted with respect to secondary Y-axis. An initial burst release of 1.12+0.3 ug on the first hour was followed by a sustained release of 0.24+0.1, 0.18+0.1, 0.05+0.01, and 0.03+0.01 ug on hours 3, 6, 12, and 24, respectively. After day-1, an amount closer to 30 ng was sustained release between every two time points that were used in the study for up to 14 days, and a 80 ng of paclitaxel was released between day-14 and day-28.

FIG. 13C shows the cumulative nitric oxide release profile for the stent of Example 3 (coated on the luminal surface with DETA NONOate). All the nitric oxide coating was burst released by the hour-1.

FIGS. 13D and E show the paclitaxel release profile and the amount of paclitaxel released between every two consecutive time points for the co-coated stent of Example 5, respectively. FIG. 13F shows the nitric oxide release profile the co-coated stent. Similar to the single drug coated stents of Examples 2 and 3, the paclitaxel showed a biphasic drug release profile with an initial burst in the first hour followed by a sustained release for up to 28 days while the nitric oxide was burst released in the first hour. Thus, the paclitaxel and nitric oxide were co-delivered from the abluminal and luminal surfaces of the stent, respectively.

### Example Nine

In Example 9, Co—Cr alloy samples were coated with either polyethylene oxide (“PEO”) alone (i.e., without incorporating paclitaxel) or with a PEO coating containing varying concentrations of paclitaxel.

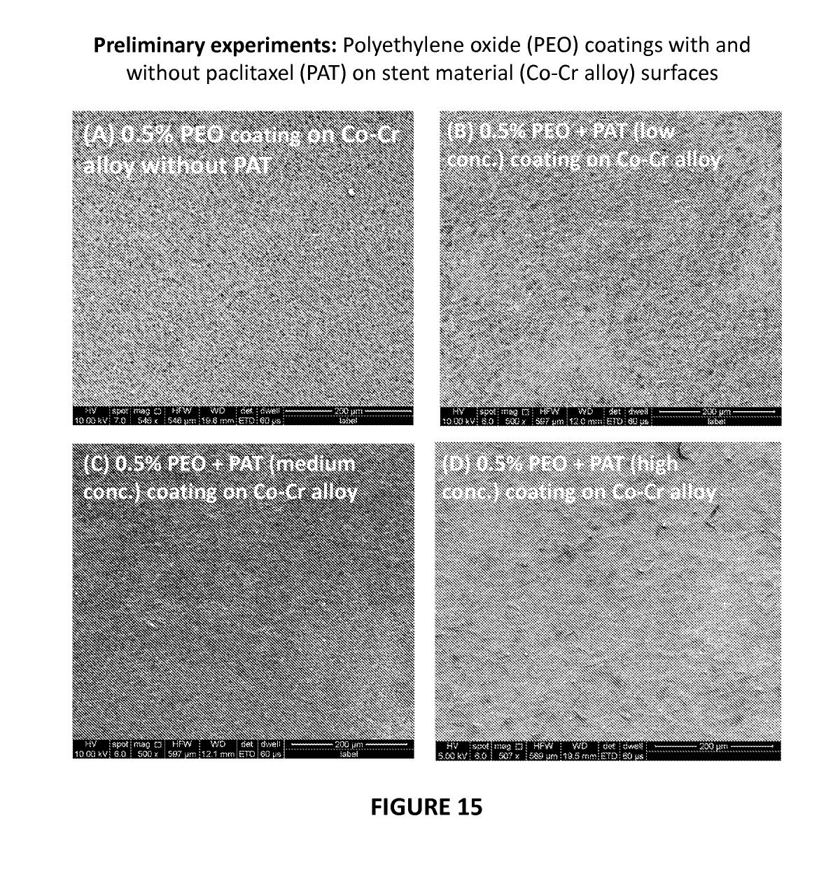

FIG. 15A is an SEM image showing the Co—Cr alloy coated with PEO alone (i.e., without incorporating paclitaxel). FIG. 15B shows the Co—Cr alloy surface coated with PEO containing a low concentration (1 mg/mL) of paclitaxel. FIG. 15C shows the Co—Cr alloy surface coated with PEO containing a medium concentration (2 mg/mL) of paclitaxel. FIG. 15D shows the Co—Cr alloy surface coated with PEO containing a high concentration (4 mg/mL) of paclitaxel.

### Example Ten

In Example 10, one Co—Cr alloy was coated with heparin alone (i.e., without incorporating DETA NONOate), while another Co—Cr alloy was coated with DETA NONOate incorporated heparin.

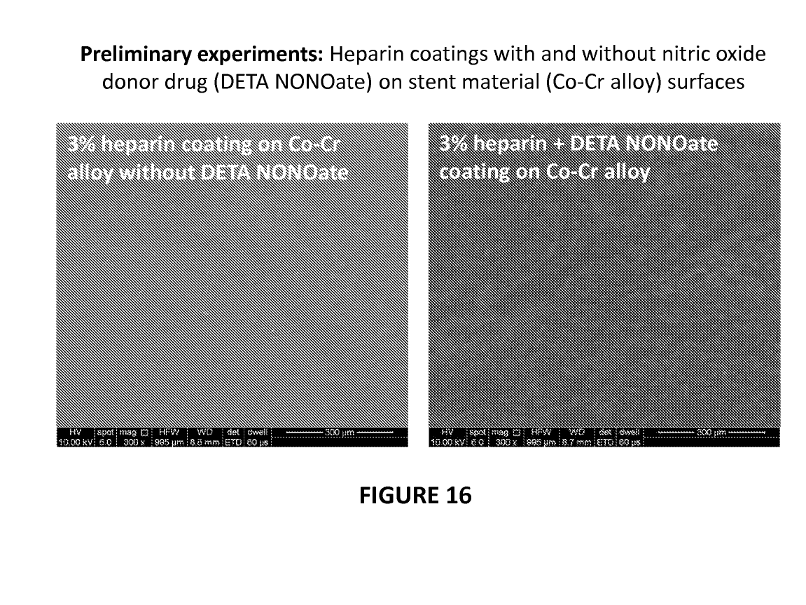

FIG. 16A is an SEM image showing the Co—Cr alloy coated with heparin alone (i.e., without incorporating DETA NONOate). FIG. 16B shows the Co—Cr alloy surface coated with DETA NONOate (2 mg/mL) incorporated heparin.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

Although the present invention has been described with reference to preferred embodiments, persons skilled in the art will recognize that changes may be made in form and detail without departing from the spirit and scope of the invention.

What is claimed is:

1. An implantable medical device comprising:
a device body having a luminal surface and an abluminal surface;
a first coating disposed on the abluminal surface, the first coating configured to elute an antiproliferative drug; and
a second coating disposed on the luminal surface, the second coating configured to elute an endothelialization promotion agent.

2. The implantable medical device of claim 1, wherein the medical device is a stent or a vascular graft.

3. The implantable medical device of claim 1, wherein the luminal surface is at least substantially free of the antiproliferative drug.

4. The implantable medical device of claim 1, wherein the abluminal surface is at least substantially free of the endothelialization promotion agent.

5. The implantable medical device of claim 1, wherein the antiproliferative drug comprises one or more of Sirolimus, Everolimus, Zotarolimus, Tacrolimus, Umirolimus, Pimecrolimus, Dexamethasone, Aspirin or paclitaxel.

6. The implantable medical device of claim 1, wherein the endothelialization promotion agent comprises a material that elutes nitric oxide.

7. The implantable medical device of claim 1, wherein the device body comprises metal, polymer or ceramic.

8. The implantable medical device of claim 1, wherein the device body comprises a cobalt chromium alloy.

9. The implantable medical device of claim 1, wherein the endothelialization promotion agent comprises nitric oxide.

10. A method of controlling neointimal hyperplasia and encouraging growth of endothelial cells on outer and inner surfaces of the stent, respectively, the method comprising:
implanting a stent having a first therapeutic agent on an outer surface of the stent and a second therapeutic agent on an inner surface of the stent;
eluting the first therapeutic agent from the outer surface of the stent; and
eluting the second therapeutic agent from the inner surface of the stent.

11. The method of claim 10, wherein eluting the first therapeutic agent comprises eluting only from the outer surface of the stent.

12. The method of claim 10, wherein eluting the second therapeutic agent comprises eluting only from the inner surface of the stent.

13. The method of claim 10, wherein eluting the first therapeutic agent comprises eluting paclitaxel.

14. The method of claim 10, wherein eluting the second therapeutic agent comprises eluting nitric oxide.

15. A method of forming a directional eluting stent having an inner surface and an outer surface, the method comprising:
providing a stent having an inner surface and an outer surface;
coating a paclitaxel-containing polymer onto the outer surface of the stent; and
coating a nitric oxide donor-containing polymer onto the inner surface of the stent.

16. The method of claim 15, wherein providing a stent comprises providing a metallic stent.

17. The method of claim 15, wherein providing a stent comprises providing a cobalt chromium alloy stent.

18. The method of claim 15, further comprising:
first contacting the inner and outer surfaces of the stent with phosphonoacetic acid prior to the coating steps; and
masking the outer surface of the stent,
wherein the coating the nitric oxide donor-containing polymer further comprises spraying the nitric oxide donor-containing polymer onto the inner surface of the stent after masking the outer surface, the nitric oxide donor drug including groups that form electrostatic interactions with the phosphonoacetic acid, and
wherein the coating a paclitaxel-containing polymer further comprises spraying the paclitaxel-containing polymer onto the outer surface of the stent, the paclitaxel including hydroxyl groups that bond with the phosphonoacetic acid.

19. The method of claim 15, further comprising:
first contacting the inner and outer surfaces of the stent with phosphonoacetic acid prior to the coating steps; and
coating a mandrel with a nitric oxide donor,
wherein the coating the paclitaxel-containing polymer further comprises spraying the paclitaxel-containing polymer onto the outer surface of the stent, the paclitaxel bonding with the phosphonoacetic acid, and
wherein the coating the nitric oxide donor-containing polymer further comprises placing the stent on the mandrel to transfer the nitric oxide donor-containing polymer from the mandrel to the inner surface of the stent, the nitric oxide donor-containing polymer including groups that form electrostatic interactions with the phosphonoacetic acid.
